(19) United States
(12) Patent Application Publication
(10) Pub. No.: US 2021/0169381 A1
(43) Pub. Date: Jun. 10, 2021

(54) WEARABLE MEASURING DEVICE AND METHOD FOR MEASURING BIOLOGICAL TARGETS USING THE SAME

(71) Applicant: Industrial Technology Research Institute, Hsinchu (TW)

(72) Inventors: Yi-Ting TUNG, Tainan City (TW); Chuhsuan CHEN, Zhubei City (TW)

(73) Assignee: Industrial Technology Research Institute, Hsinchu (TW)

(21) Appl. No.: 17/109,905

(22) Filed: Dec. 2, 2020

Related U.S. Application Data
(60) Provisional application No. 62/945,433, filed on Dec. 9, 2019.

(30) Foreign Application Priority Data
Jun. 16, 2020 (TW) 109120259

(51) Int. Cl. A61B 5/1486; A61B 5/00; A61B 5/1495

(52) U.S. Cl. CPC A61B 5/1486; A61B 5/1495; A61B 5/6801

(57) ABSTRACT

A wearable measuring device is provided. The wearable measuring device includes a wearable element, a liquid-transferring element, a bio-sensing electrode, a flow-sensing electrode, and a meter. The wearable element has an opening. The liquid-transferring element is disposed in the wearable element and has a sampling portion exposed to the outside through the opening. The bio-sensing electrode and the flow-sensing electrode are connected to a first position and a second position of the liquid-transferring element. The meter is disposed on or in the wearable element and is electrically connected to the bio-sensing electrode and the flow-sensing electrode.

### CROSS REFERENCE TO RELATED APPLICATION

This application claims the benefit of U.S. Provisional Application No. 62/945,433, filed Dec. 9, 2019, and Taiwan Patent Application No. 109120259, filed Jun. 16, 2020, the entirety of which is incorporated by reference herein.

### TECHNICAL FIELD

The technical field relates to a measuring device and a method for measuring biological targets, and in particular to a wearable measuring device and a method for measuring biological targets using the same.

### BACKGROUND

Development of a wearable, continuous measuring device has been a goal pursued by people engaged in developing new measuring devices. No matter whether optical measurements or electrochemical measurements are made, direct contact between a sample and the measurement instrument is necessary when using existing wearable, continuous measuring devices. Elements such as microneedles and subcutaneous implants are used to minimize users' discomfort caused by the direct contact between the sample and the measurement. Although efforts are made to minimize this discomfort, most users still feel discomfort during physical activity.

Therefore, there is a great need for a novel, wearable measuring device to solve the discomfort caused by direct contact between the users' skin and the measuring device during physical activity.

### SUMMARY

In the wearable measuring device of the embodiments of the present disclosure, a liquid-transferring element is set as a medium between a sensing electrode and the users' skin to avoid the discomfort caused by the direct contact between the users' skin and the sensing electrode.

An embodiment of the present disclosure provides a wearable measuring device. The wearable measuring device includes a wearable element, a liquid-transferring element, a bio-sensing electrode, a flow-sensing electrode and a meter. The wearable element includes an opening. The liquid-transferring element is disposed in the wearable element. The liquid-transferring element includes a sampling portion exposed outside through the opening. The bio-sensing electrode and the flow-sensing electrode are connected to a first position and a second position of the liquid-transferring element respectively. The meter is disposed on or in the wearable element and electrically connected to the bio-sensing electrode and the flow-sensing electrode.

An embodiment of the present disclosure provides a method for measuring biological targets. The method includes the following steps: continuously measuring an instantaneous biological target concentration in the biological liquid using the bio-sensing electrode; continuously measuring an instantaneous biological liquid volume using a flow-sensing electrode; and calibrating a biological target concentration using the following formula:

$${\sum }_{n = 0}^{t} fn ( An ) fn ( Sn ) + c ,$$

wherein An represents the instantaneous biological target concentration in the biological liquid, Sn represents the instantaneous biological liquid volume, t represents a measuring time, and c is a constant.

### BRIEF DESCRIPTION OF DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity illustrating the features of the embodiment of the present disclosure.

FIG. 1 is a plan view of a wearable measuring device according to an embodiment of the present disclosure.

FIGS. 2A-2C are schematic views of a bio-sensing electrode according to some embodiments of the present disclosure.

FIG. 3A is a response current signal change graph of a bio-sensing electrode with a reacting element of the present disclosure during measuring the biological targets with Amperometry (IT).

FIG. 3B is a response current signal change graph of an electrode without any reacting element during measuring the biological targets with Amperometry (IT).

FIG. 4 is a signal change graph of bio-sensing electrodes according to some embodiments of the present disclosure detected by Amperometry (IT) with an electrochemical meter.

FIGS. 5A-5F are schematic views of configurations of a flow-sensing electrode according to some embodiments of the present disclosure.

FIG. 6A is a schematic view of configurations of a flow-sensing electrode according to other embodiments of the present disclosure.

FIG. 6B is a signal change graph of the flow-sensing electrode with the configuration shown in FIG. 6A.

FIG. 7 is a plan view of a wearable measuring device according to another embodiment of the present disclosure.

FIG. 8 is an operation flow chart of the wearable measuring device shown in FIG. 7.

### DETAILED DESCRIPTION

The following disclosure provides many different implementations or examples for practicing different features of the disclosure. Specific examples of components and configurations thereof are described below to illustrate the embodiments of the present disclosure. These are, of course, merely examples and are not intended to be limiting. For example, the formation of a first feature on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various embodiments. This repetition is for the purpose of simplicity and clarity and does not in itself dictate a specific relationship between the various embodiments and/or structures discussed.

In addition, spatially relative terms, for example, “beneath,” “below,” “lower,” “upper,” “above,” “over,” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terms “about,” “approximate” and “substantially” typically mean +/−20%, preferably +/−10%, more preferably +/−5%, or +/−3%, or +/−2%, or +/−1%, or +/−0.5% of the stated value. It should be noted that the stated value of the present disclosure is an approximate value. When there is no specific description, the stated value includes the meaning of “about,” “approximate” and “substantially”.

It should be understood that, although the terms first, second, third etc. may be used herein to describe various elements, components, regions, layers, portions, and/or sections, these elements, components, regions, layers, portions, and/or sections should not be limited by these terms. These terms are only used to distinguish one element, component, region, layer, portion, and/or section from another element, component, region, layer, portion, and/or section. Thus, a first element, component, region, layer, portion, and/or section discussed below could be termed a second element, component, region, layer, portion, and/or section without departing from the teachings of the present disclosure.

Although the steps in some of the described embodiments are performed in a specific order, these steps can also be performed in other logical orders. In different embodiments, some of the steps described may be replaced or omitted, and some other operations may be performed before, during, and/or after the steps described in the embodiments of the present disclosure.

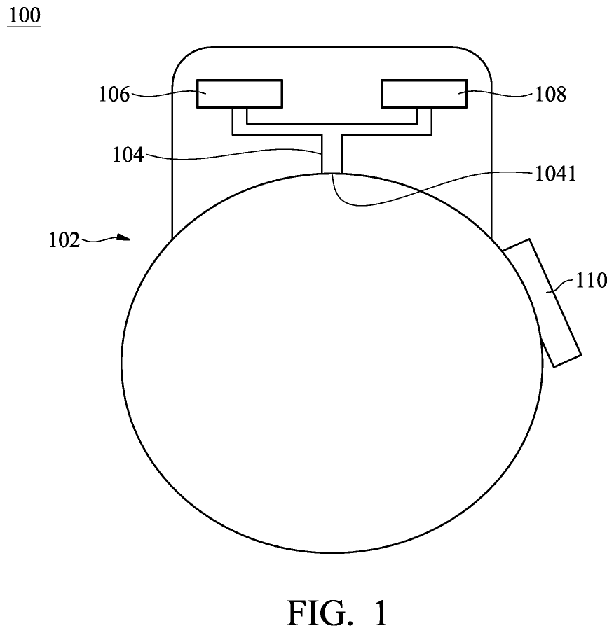

Referring to FIG. 1, FIG. 1 is a plan view of a wearable measuring device 100 according to an embodiment of the present disclosure. The wearable measuring device 100 includes a wearable element 102, a liquid-transferring element 104, a bio-sensing electrode 106, a flow-sensing electrode 108, and a meter 110.

The wearable element 102 includes an opening. In particular, the opening of the wearable element 102 faces the users' skin. In some embodiments, the wearable element 102 includes hats, headbands, wristbands, clothes, or the like. The opening may be in, for example, the hat body of the hat, the inside of the headband, the cuffs or collar of the clothes, etc.

The liquid-transferring element 104 may be disposed in the wearable element 102. The liquid-transferring element 104 may have a sampling portion 1041. The sampling portion 1041 may be exposed to the outside to make contact with the users' skin through the opening of the wearable element 102. A biological liquid from the users' skin may enter to the liquid-transferring element 104 through the sampling portion 1041. The liquid-transferring element 104 may include a single-layer or multi-layer structure, and may be semicircular, circular, square, rectangular, L-shaped, T-shaped, or Y-shaped, but it is not limited thereto. The liquid-transferring element 104 may include a pump or an absorbent material. Examples of the absorbent material include superabsorbent polymers (SAP), hydrophilic fiber materials, and combinations thereof. In an embodiment in which a pump is used as the liquid-transferring element 104, the biological liquid could be moved in the liquid-transferring element 104 by the pump after entry to the liquid-transferring element 104. In an embodiment in which an absorbent material is used as the liquid-transferring element 104, the biological liquid could be moved in the liquid-transferring element 104 by a capillary action of the absorbent material after entry to the liquid-transferring element 104. In some embodiments, examples of the biological liquid include sweat, blood or the like.

The bio-sensing electrode 106 may be an electrode used for measuring the biological target concentration in the biological liquid. Examples of the biological targets (or biochemical substances, metabolites, etc.) may include electrolytes, glucoses, lactic acids or the like. The flow-sensing electrode 108 may be an electrode used for measuring the biological liquid volume. The bio-sensing electrode 106 and the flow-sensing electrode 108 are disposed in the wearable element 102 and connected to the liquid-transferring element 104. The biological liquid could be moved to the bio-sensing electrode 106 and the flow-sensing electrode 108 by said connections while the bio-sensing electrode 106 and flow-sensing electrode 108 did not make contact with the users' skin. Therefore, the users' discomfort could be relieved by reducing the direct contact between the bio-sensing electrode 106 and/or the flow-sensing electrode 108 and the users' skin. The bio-sensing electrode 106 and the flow-sensing electrode 108 may be connected to any position of the liquid-transferring element 104. For example, as shown in FIG. 1, in cases where the liquid-transferring element 104 is a Y-shaped liquid-transferring element with a first end, a second end, and a third end, the sampling portion 1041 may be at the first end of the liquid-transferring element 104. The bio-sensing electrode 106 and the flow-sensing electrode 108 may be disposed near the second end and the third end of the liquid-transferring element 104 respectively. In the embodiment, in the wearable measuring device 100, the distance between the bio-sensing electrode 106 and the sampling portion 1041 may be substantially the same as the distance between the flow-sensing electrode 108 and the sampling portion 1041. Therefore, the biological liquid may reach the bio-sensing electrode 106 and the flow-sensing electrode 108 at substantially the same time, but it is not limited thereto.

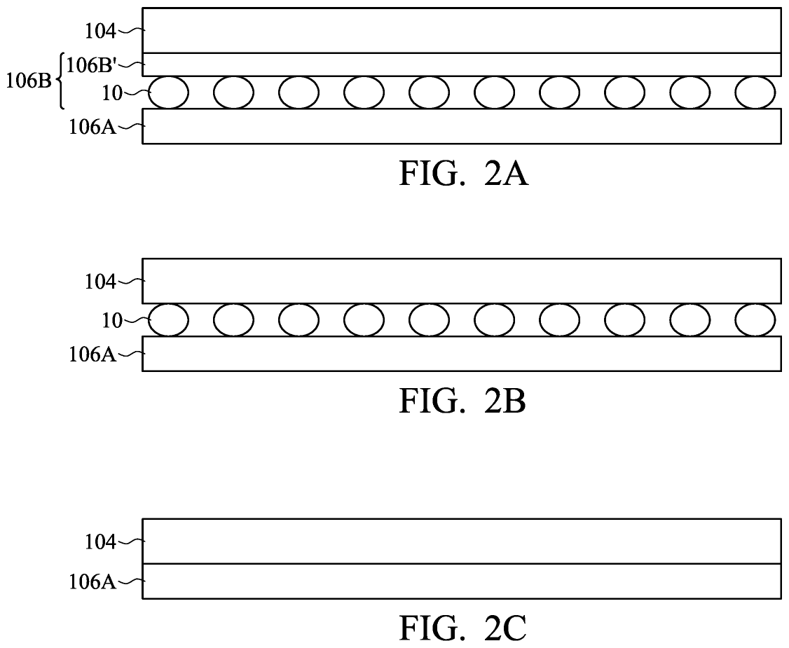

FIGS. 2A-2C are schematic views of a bio-sensing electrode 106 according to some embodiments of the present disclosure. The bio-sensing electrode 106 includes a conductive element 106A. The conductive element 106A may include an electrode or a conductive webbing. The electrode includes conductive materials, and the conductive webbing is woven from conductive materials (conductive woven threads) and artificial fibers or natural fibers. In an embodiment, the conductive element 106A may directly contact with the liquid-transferring element 104 so as to react with the biological targets in the biological liquid that enters the liquid-transferring element 104, as shown in FIG. 2C. In this embodiment, the conductive element 106A may include a conductive material selected from cobalt, nickel, selenium, cobalt oxide, nickel oxide, selenium oxide, silver carbon, graphene, carbon nanotube, or any combination thereof. The conductive material mentioned above may be used as the electrode or the conductive woven threads of the conductive webbing. In another embodiment, the bio-sensing electrode 106 may further include a reacting element 106B. The reacting element 106B includes reactants 10 and a fixing layer 106B′. The reacting element 106B may be disposed between the conductive element 106A and the liquid-transferring element 104 as shown in FIG. 2A. The reacting element 106B may directly contact with the liquid-transferring element 104 so as to react with the biological targets in the biological liquid that enters the liquid-transferring element 104. The conductive element 106A may generate an electrical signal in response to a reaction between the reacting element 106B and the biological targets. In such embodiments, the conductive element 106A may include conductive materials, such as aluminum (Al), molybdenum (Mo), tungsten (W), titanium (Ti), tantalum (Ta), platinum (Pt), hafnium (Hf), nickel (Ni), cobalt (Co), zinc (Zn), graphite, or the like, but it is not limited thereto. The reactants 10 may include immobilized enzymes, enzymes, antibodies, proteins, nucleic acids, chemical reaction reagents or a combination thereof, but they are not limited thereto. For example, the reactants 10 may include glucose oxidase, glucose dehydrogenase, lactate oxidase, lactate dehydrogenase, 3-hydroxybutyrate dehydrogenase, urease, cholesterol esterase, cholesterol oxidase, or any combination thereof, but they are not limited thereto. A person having ordinary skill in the art may understand that types of reactants will vary with the biological targets to be measured.

The reactants 10 may be affixed to the conductive element 106A in various ways. In an embodiment, as shown in FIG. 2, the reacting element 106B may further include the fixing layer 106B′ to fix the reactants 10 on the conductive element 106A. The materials of the fixing layer 106B′ may include poly vinyl pyrrolidone (PVP), poly ethylene glycol (PEG), poly ethylene imine (PEI), polyvinyl alcohol (PVA) or carboxymethyl cellulose (CMC). In one embodiment, the fixing layer 106B′ may be disposed on the reactants 10. In other embodiment, the fixing layer 106B′ may be disposed between the reactants 10 and the conductive element 106A. In other embodiment, the reactants 10 may be disposed between two fixing layers 106B′.

In another embodiment, for example, in cases where that the reactants 10 are immobilized enzymes, the reactants 10 may be affixed to the conductive element 106A without the fixing layer, as shown in FIG. 2B. For example, the reactants 10 may be affixed directly to the conductive element 106A using 1-ethyl-3-(3-dimethylaminopropyl) (EDC)/N-hydroxysuccinimide (NHS), EDC/sulfo-NHS, glutaraldehyde (GA), Nafion or an electrochemical method.

The bio-sensing electrode 106 may be used for measuring the biological target concentration in the biological liquid by an electrochemical method, such as a general current and voltage method, Amperometry (IT), Differential pulse voltammetry (DPV) or Square wave voltammetry (SWV), etc.

Differential pulse voltammetry is a measuring method superimposing a certain amplitude pulse voltage (for example, 10-100 mV) on a direct current voltage to make a measurement. Square wave voltammetry (SWV) is a method applying a square wave potential of a large amplitude differential pulse to the electrode to obtain two response current after a square wave cycle period. One of the two response currents is a current at the end of a forward potential pulse and another is a current at the end of a reverse potential pulse. A net current could be obtained by subtracting the current at the end of the forward potential pulse from the current at the end of the reverse potential pulse. Square wave voltammetry has an effect of amplifying signals, and has better sensitivity than Differential pulse voltammetry. In addition, other advantage of Square wave voltammetry includes fast scanning. The effective scanning rate is the square wave frequency multiplied by the potential change, so that the scanning time could be reduced and the analysis could be completed quickly.

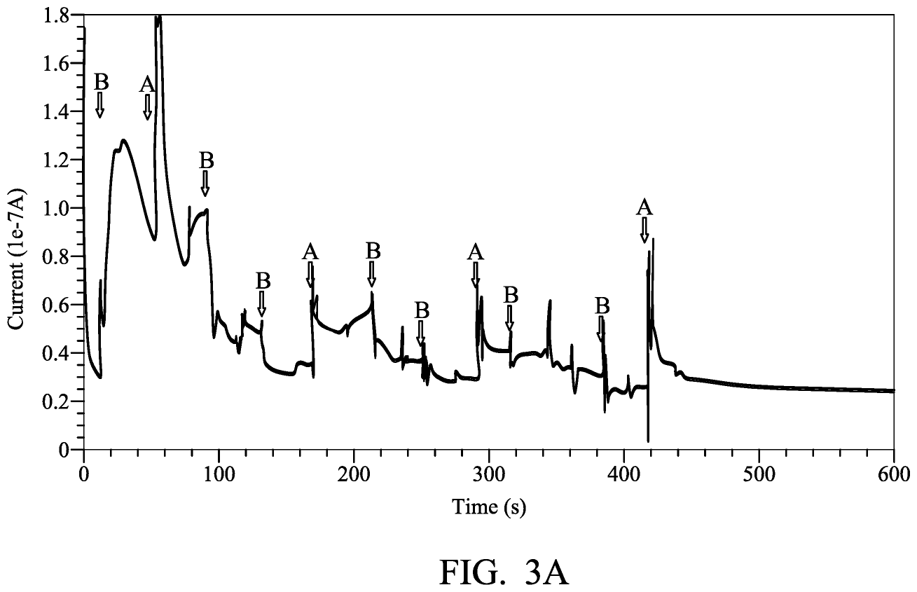

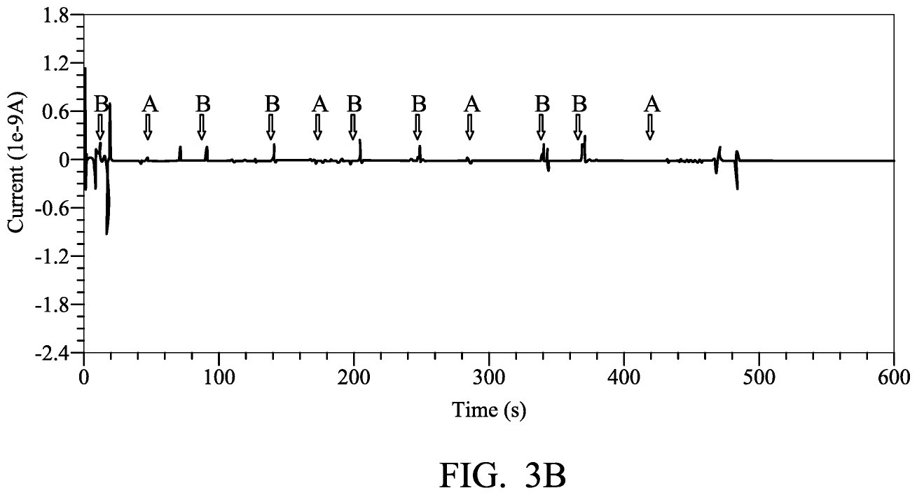

FIG. 3A is a response current signal change graph of a bio-sensing electrode 106 with a reacting element 106B of the present disclosure during measuring the biological targets with Amperometry (IT). FIG. 3B is a response current signal change graph of an electrode without any reacting element during measuring the biological targets with Amperometry (IT).

In FIG. 3A, for example, a filter paper is used as the liquid-transferring element 104, lactic acid is used as the biological targets and a gold film electrode with lactate oxidase is used as the bio-sensing electrode 106. In this embodiment, the lactate oxidase is affixed to the gold film electrode by EDC/NHS, and the lactate oxidase is used as the reacting element 106B as shown in FIG. 2A. In FIG. 3A, arrow marks “A” indicate the injections of 100 mL of lactic acid, and arrow marks “B” indicate the injections of PBS (phosphate buffered solution). As shown in FIG. 3A, the vertical axis refers to equilibrium response current (1e-7 ampere in unit) and the horizontal axis refers to time (sec). The equilibrium response current will rise when the lactic acid is contact with the bio-sensing electrode 106, and the equilibrium response current will drop when the PBS is contact with the bio-sensing electrode 106. In FIG. 3A, in response to the injections of the lactic acid, the response current rises from 1.8×10⁻⁸ A to 3.5×10⁻⁸ A when the time is from 160 sec to 180 sec, and the response current rises from 1.2×10⁻⁸ A to 2.5×10⁻⁸ A when the time is from 290 sec to 310 sec. Further, in response to the injections of the PBS, the response current drops from 4.0×10⁻⁸ A to 1.2×10⁻⁸ A when the time is from 200 sec to 260 sec, and drops from 2.5×10⁻⁸ A to 5.7×10⁻⁹ A when the time is from 310 sec to 400 sec. The lactic acid content can be estimated by the change of the equilibrium response current.

In FIG. 3B, for example, a filter paper is used as the liquid-transferring element 104, lactic acid is used as the biological targets and a gold film electrode is used as the bio-sensing electrode 106 without the lactate oxidase used as the reacting element 106B. The vertical axis refers to equilibrium response current (1e-9 ampere in unit) and the horizontal axis refers to time (sec). In FIG. 3B, arrow marks “A” indicate the injections of 100 mL of lactic acid, and arrow marks “B” indicate the injections of PBS. As shown in FIG. 3B, no reaction response current is generated no matter whether the lactic acid is injected or not. There is only background current noise in FIG. 3B.

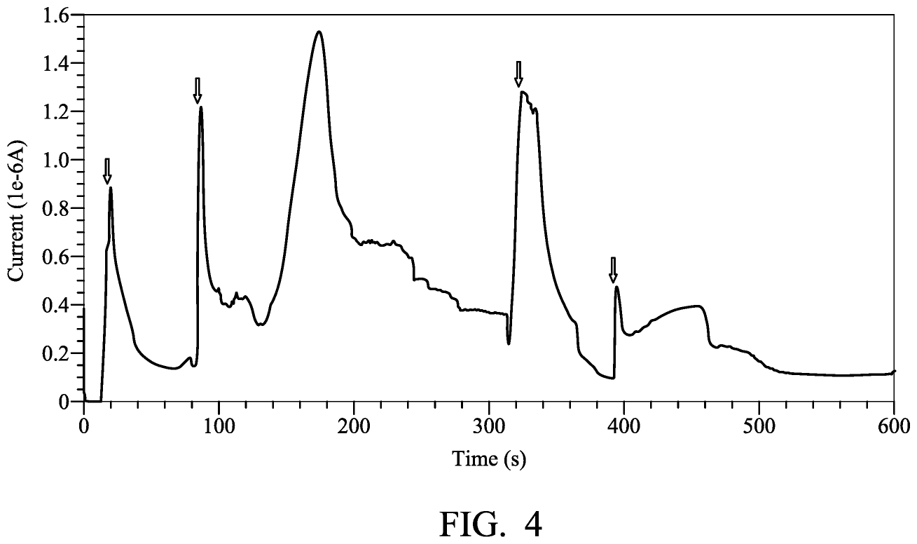

FIG. 4 is a response current signal change graph of the bio-sensing electrode 106 of the wearable measuring device 100 during measuring the biological targets with Amperometry (IT) for 10 minutes. The vertical axis refers to equilibrium response current (1e-6 ampere in unit) and the horizontal axis refers to time (sec). In FIG. 4, for example, an absorbent material is used as the liquid-transferring element 104, glucose is used as the biological targets, and glucose oxidase is used as the reactants 10. The glucose oxidase are directly immobilized on the conductive element 106A so that to complete the bio-sensing electrode 106 as shown in FIG. 2B.

As shown in FIG. 1, the liquid-transferring element 104 is a Y-shaped liquid-transferring element with a first end, a second end, and a third end. The first end of the Y-shaped liquid-transferring element 104 is used as the sampling portion 1041, and the second end of the Y-shaped liquid-transferring element 104 opposed to the sampling portion 1041 is connected with the bio-sensing electrode 106, as shown in FIG. 1. A glucose solution is added to the sampling portion 1041 in several times. The glucose solution may move in the liquid-transferring element 104 and pass through a reacting region of the bio-sensing electrode 106 by the absorption and/or pipetting function of the liquid-transferring element 104. Response current signals may be collected from the bio-sensing electrode 106, and the result is shown in FIG. 4. According to FIG. 4, it could be found that peaks could be detected (as shown by the arrow) while the glucose solution is added. The current value of the peak could be converted to the glucose concentration. After the glucose solution is added, the response current will rise rapidly at first, and then be stable at a higher response current value. By recording the stable response current value and matching it with a table established with standard products, the glucose concentration in a sample could be obtained. The biological liquid could be continuously injected to the reacting region of the bio-sensing electrode 106 by the pipetting function of the absorbent material to conduct an electrochemical reaction, and the wearable measuring device 100 could continuously and repeatedly measure the biological target concentration in the sample.

FIGS. 4A-4F are schematic views of configurations of the flow-sensing electrode 108 and the liquid-transferring element 104 according to some embodiments of the present disclosure. The flow-sensing electrode 108 may include conductive components. The conductive components may include an electrode or conductive webbing for measuring a small volume change of the biological liquid. The electrode may include conductive materials, and the conductive webbing may be woven from conductive materials and artificial fibers or natural fibers. Examples of the conductive materials include aluminum (Al), molybdenum (Mo), tungsten (W), titanium (Ti), tantalum (Ta), platinum (Pt), hafnium (Hf), nickel (Ni), cobalt (Co), zinc (Zn), and graphite.

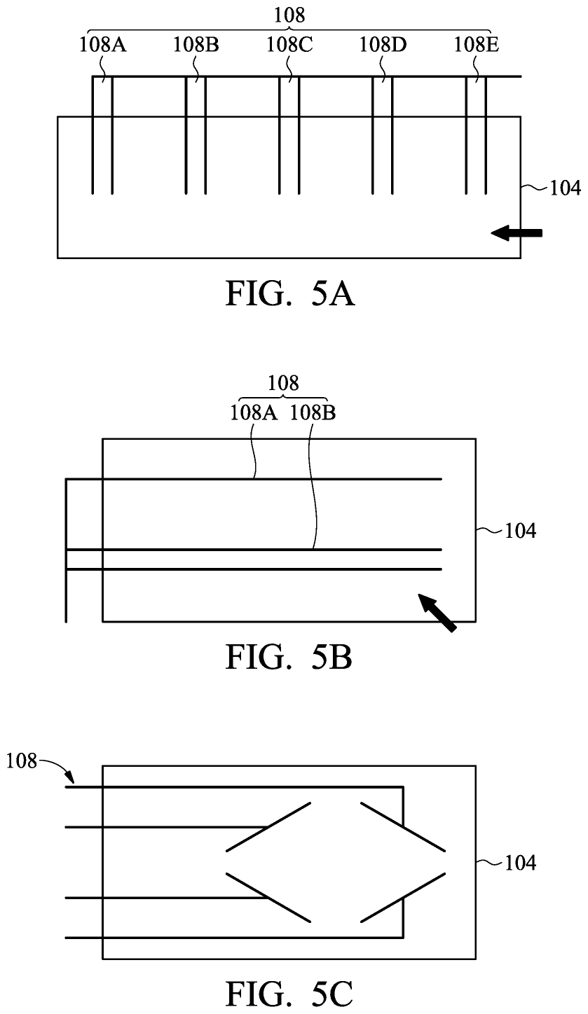

In an embodiment, the flow-sensing electrode 108 may include more than two conductive components. As shown in FIG. 5A, the flow-sensing electrode 108 connected to the liquid-transferring element 104 may include a first conductive component 108A, a second conductive component 108B, a third conductive component 108C, a fourth conductive component 108D, and a fifth conductive component 108E. The distance between the first conductive component 108A and the sampling portion is greater than the distance between the second conductive component 108B and the sampling portion, the distance between the second conductive component 108B and the sampling portion is greater than the distance between the third conductive component 108C and the sampling portion, the distance between the third conductive component 108C and the sampling portion is greater than the distance between the fourth conductive component 108D and the sampling portion, and the distance between the fourth conductive component 108D and the sampling portion is greater than the distance between the fifth conductive component 108E and the sampling portion. Each of the conductive components may include a double wire electrode to measure the potential difference. Distances between the conductive components are constant. The moving direction of the biological liquid is indicated by the arrow in FIG. 5A. Therefore, the biological liquid will reach the fifth conductive component 108E, the fourth conductive component 108D, the third conductive component 108C, the second conductive component 108B, and the first conductive component 108A in the aforementioned order. In such an embodiment, the fifth conductive component 108E will measure the current and voltage changes first, and then the fourth conductive component 108D to the first conductive component 108A will measure the current and voltage changes in order. Using said arrangement of the conductive components, the flow-sensing electrode 108 could measure a large change in the biological liquid volume. A precise total biological liquid or water volume in the biological liquid (also known as water content, moisture content) could be obtained by measuring the change in the biological liquid volume using the first conductive component 108A to the fifth conductive component 108E respectively.

In another embodiment, as shown in FIG. 5B, the flow-sensing electrode 108 connected to the liquid-transferring element 104 may include a first conductive component 108A (a single-wire electrode) and a second conductive component 108B (a double-wire electrode). The measurement accuracy of the change in the biological liquid volume could be improved by increasing exposed sensing areas of the first conductive component 108A and the second conductive component 108B, which are contacted with the biological liquid. In the embodiment of FIG. 5B, the biological liquid enters from the arrow marked site. When the biological liquid volume increases, the current and voltage changes measured by the second conductive component 108B became greater. When the biological liquid volume exceeds a certain level, the biological liquid will move to the first conductive component 108A, and the first conductive component 108A starts to measure the current and voltage changes. Using said arrangement of the conductive components, the flow-sensing electrode 108 can measure a large change in the biological liquid volume. A precise total biological liquid volume or water content could be obtained by the measured change in the biological liquid volume using the first conductive component 108A and the second conductive component 108B.

The arrangement of the conductive components of the flow-sensing electrode 108 could be adjusted as needed. For example, the conductive components of the flow-sensing electrode 108 could be geometrically arranged as shown in FIG. 5C (4 straight line electrodes facing each other), palisade arranged as shown in FIG. 5D (2 finger electrodes interlacing with each other) and 5E or S-shaped crossing arranged as shown in FIG. 5F (they overlap in space but do not contact each other), but they are not limited thereto. In addition to collecting current and/or voltage changes, the measurement could also be performed using electrochemical methods such as DVP and SWV.

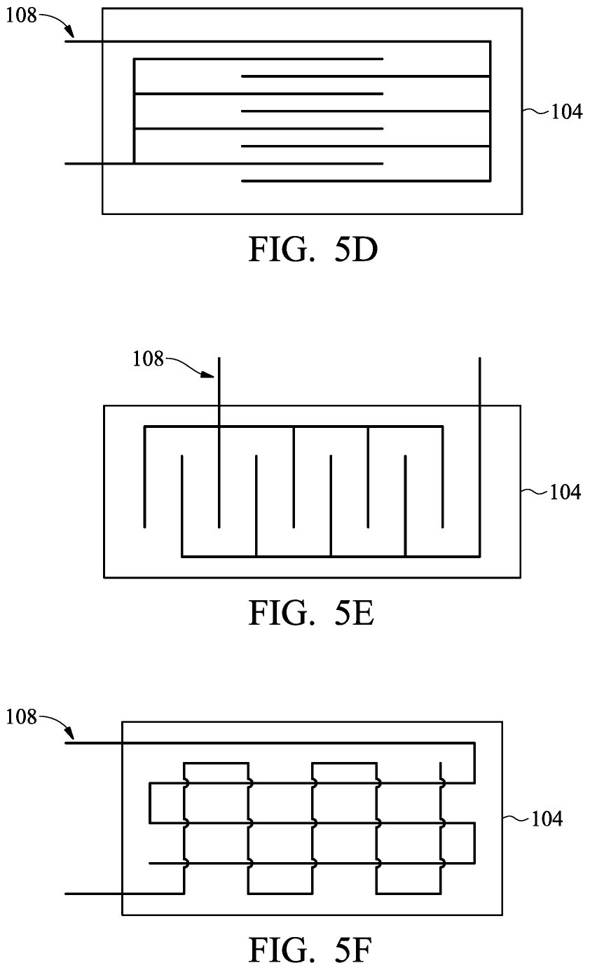

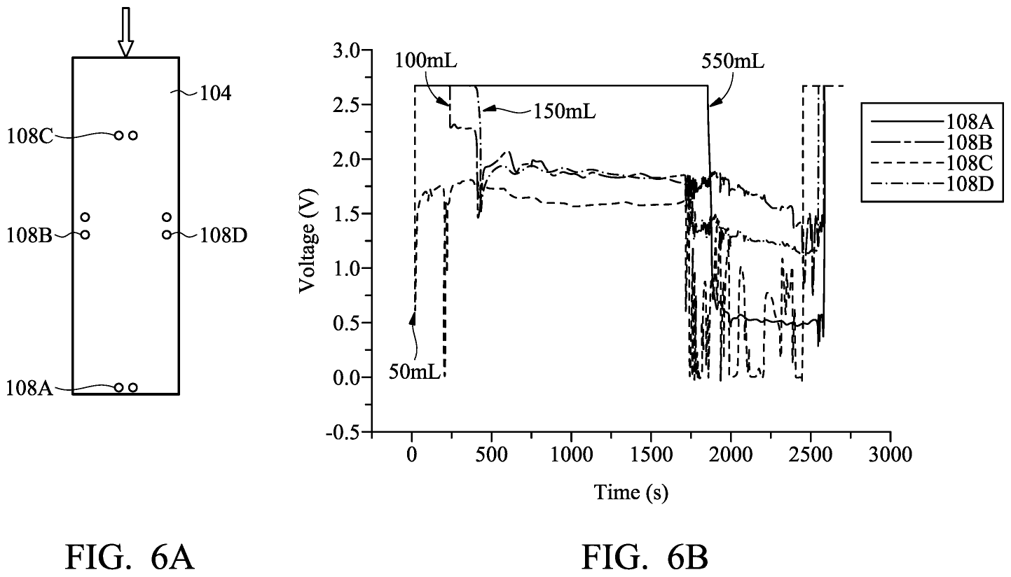

The measurement principle of the flow-sensing electrode 108 is described in further detail below. FIG. 6A is a schematic view of a configuration of flow-sensing electrodes 108 according to other embodiments of the present disclosure. As shown in FIG. 6A, the flow-sensing electrode 108 connected to the liquid-transferring element 104 includes a first conductive component 108A, a second conductive component 108B, a third conductive component 108C, and a fourth conductive component 108D (to simplify the description, two dots are used to indicate the exposed sensing area of each conductive component or point electrodes in FIG. 6A). The moving direction of the biological liquid is indicated by the arrow shown in FIG. 6A. The first conductive component 108A is away from the sampling portion, the third conductive component 108C is closest to the sampling portion, the second and the fourth conductive component 108B and 108D are between the first conductive component 108A and the third conductive component 108C.

FIG. 6B is a signal change graph of the flow-sensing electrode 108 with the configuration shown in FIG. 6A. A phosphate buffer solution (PBS) is added into the sampling portion every three minutes. The third conductive component 108C will respond to the addition of the PBS first because the third conductive component 108C is closest to the sampling portion. As shown in FIG. 6B, the voltage of the third conductive component 108C drops when the volume of the PBS is accumulated up to 50 mL. The voltage drop represents that the potential of PBS has been measured. The second and the fourth conductive component 108B and 108D are farther away from the sampling portion than the third conductive component 108C. Therefore, the second and the fourth conductive component 108B and 108D will respond to the addition of the PBS after the responses of the third conductive component 108C. As shown in FIG. 6B, when the volume of the PBS is accumulated up to 100 mL and 150 mL, the voltage of the second and the fourth conductive component 108B and 108D drop respectively. The first conductive component 108A will respond at last because the first conductive component 108A is at a position farthest away from the sampling portion. As shown in FIG. 6B, the voltage of the first conductive component 108A drops when the volume of the PBS is accumulated up to 550 mL. Therefore, water in the biological liquid could be measured by the arrangement of the conductive components.

Referring back to FIG. 1, the meter 110 may be disposed on or in the wearable element 102. The meter 110 could be electrically connected to the bio-sensing electrode 106 and the flow-sensing electrode 108 to receive electrical signals from the bio-sensing electrode 106 and the flow-sensing electrode 108. According to the position where the bio-sensing electrode 106 and the flow-sensing electrode 108 are connected to the liquid-transferring element 104, the electrical signals from the bio-sensing electrode 106 and the flow-sensing electrode 108 could reach the meter 110 simultaneously or sequentially. The meter 110 may include an arithmetic module to obtain and record the biological target concentration in the biological liquid, the total biological liquid volume, and the corrected biological target concentration in the biological liquid. In another embodiment, the meter 110 may further include a power supply module to provide power to the bio-sensing electrode 106 and the flow-sensing electrode 108. In another embodiment, the meter 110 may further include a display to display the biological target concentration in the biological liquid, the total biological liquid volume, and/or the corrected biological target concentration in the biological liquid.

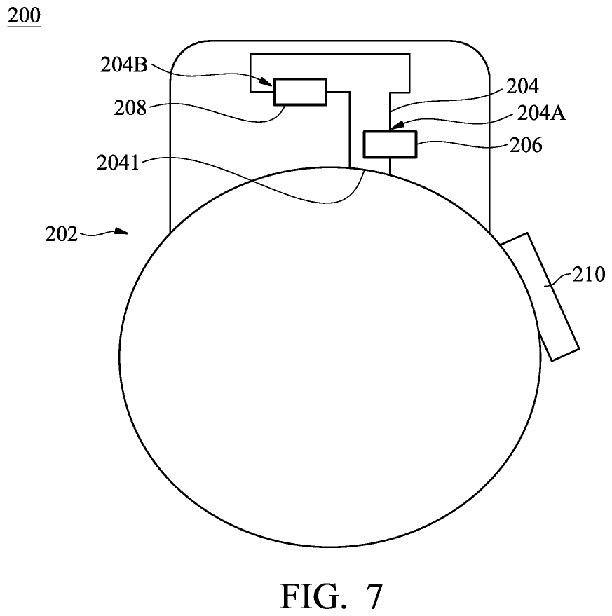

FIG. 7 illustrates a plan view of a wearable measuring device 200 according to another embodiment of the present disclosure. As shown in FIG. 7, the liquid-transferring element 204 is a L-shaped liquid-transferring element with a first end and a second end. The L-shaped liquid-transferring element 204 is disposed inside the wearable element 202, and the sampling portion 2041 is at the first end of the L-shaped liquid-transferring element 204. The bio-sensing electrode 206 and the flow-sensing electrode 208 are disposed in the wearable element 202 to be connected with the liquid-transferring element 204. As shown in FIG. 7, the bio-sensing electrode 206 and the flow-sensing electrode 208 are connected to the first position 204A and the second position 204B of the liquid-transferring element 204 respectively. The distance between the second position 204B and the sampling portion 2041 is greater than the distance between the first position 204A and the sampling portion 2041. In the wearable measuring device 200, in comparison with the flow-sensing electrode 208, the bio-sensing electrode 206 is closer to the sampling portion 2041. Therefore, the biological liquid will first reach the bio-sensing electrode 206 and then reach the flow-sensing electrode 208. The bio-sensing electrode 206 may continuously measure an instantaneous (real time) biological target concentration in the biological liquid. An instantaneous biological liquid volume could be continuously measured using flow-sensing electrode 208. In an embodiment, the flow-sensing electrode is closer to the sampling portion than the bio-sensing electrode. Therefore, the biological liquid will first reach the flow-sensing electrode and then reach the bio-sensing electrode. The structures of wearable element 202, the meter 210, the bio-sensing electrode 206, and the flow-sensing electrode 208 of the wearable measuring device 200 may be similar as that of the wearable element 102, the meter 110, the bio-sensing electrode 106, and the flow-sensing electrode 108 of the wearable measuring device 100. Therefore, the repeated description will be omitted. The wearable measuring device 200 is used as an example to explain specific operations of the wearable measuring device according to the present disclosure are described as following.

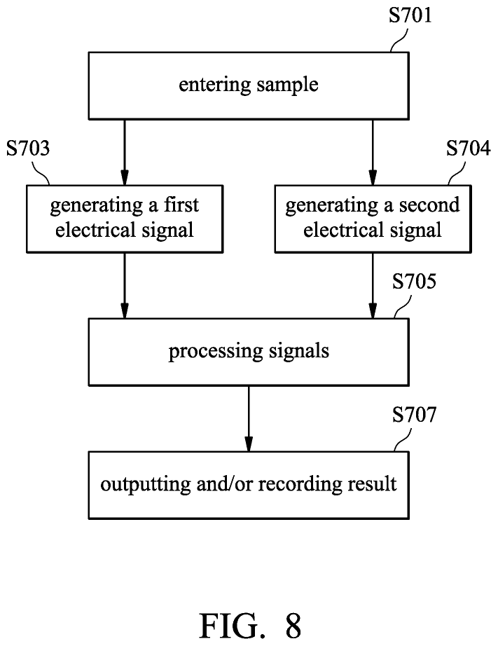

FIG. 8 is an operation flow chart of the wearable measuring device 200 according to the present disclosure. As shown in FIG. 8, the operation flow of the wearable measuring device 200 includes step of entering sample S701, step of generating a first electrical signal S703, step of generating a second electrical signal S704, step of processing signals S705, and step of outputting and/or recording result S707. Referring to FIGS. 6 and 7, in step S701, the biological liquid is absorbed and enters the L-shaped liquid-transferring element 204 through the sampling portion 2041. In the embodiment of FIG. 7, the biological liquid will move to the bio-sensing electrode 206 first, and then to the flow-sensing electrode 208. In step S703, the biological liquid will move to the bio-sensing electrode 206. The bio-sensing electrode 206 will measure the response currents while the biological liquid pass through the bio-sensing electrode 206, and generate a first electrical signal accordingly. In step S704, the biological liquid will move to the flow-sensing electrode 208. The flow-sensing electrode 208 will measure the current and voltage changes when the biological liquid pass through the flow-sensing electrode 208, and generate a second electrical signal accordingly. The perform order of step S703 and step S704 is not particularly limited, and they could be performed simultaneously or sequentially. In step S705, after the meter 210 receives the first electrical signal and the second electrical signal, the meter 210 calibrates the biological target concentration and the total biological liquid volume by the first electrical signal and the second electrical signal. The meter 210 generates the corrected biological target concentration according to the following correcting formula. In the correcting formula, “An” represents the instantaneous biological target concentration measured by the bio-sensing electrode 206, “Sn” represents the instantaneous biological liquid volume measured by the flow-sensing electrode 208, “t” represents a measuring time, and “c” is a constant. The corrected biological target concentration or the average monitoring volume can be obtained by multiplying the instantaneous biological target concentration An by the instantaneous biological liquid volume Sn, integrating the time t, and adding the constant c.

$${\sum }_{n = 0}^{t} ( fn ( An ) fn ( Sn ) ) + c$$

In step S707, the biological target concentration, the total biological liquid volume, and/or the corrected biological target concentration calibrated based on the first electrical signal and the second electrical signal are output and/or recorded, so as to provide a measured data which is continuously accumulated.

Compared with existing wearable measuring devices, the wearable measuring device according to the embodiments of the present disclosure has one or more of the following advantages, but it is not limited thereto:

(1) The wearable measuring device according to the embodiments of the present disclosure may avoid direct contact between the sensing electrode and the skin by disposing the liquid-transferring element at the opening of the wearable element. Therefore, the discomfort caused by direct contact between the sensing electrode and the skin can be avoided.
(2) The biological liquid can be continuously transported to the bio-sensing electrode and the flow-sensing electrode by the liquid-transferring element. Therefore, the wearable measuring device according to the embodiments of the present disclosure may continuously measure the biological target concentration in the biological liquid and the biological liquid volume.
(3) The liquid-transferring element, the bio-sensing electrode and the flow-sensing electrode are disposable modules. Therefore, the liquid-transferring element, the bio-sensing electrode, and the flow-sensing electrode can be changed according to different biological targets.
(4) Furthermore, by correcting the biological target concentration in the biological liquid with the water content, physiological conditions of the user could be reflected more accurately.

Although some embodiments of the present disclosure and their advantages have been described above, it should be understood that various changes, substitutions and modifications can be made herein without departing from the spirit and scope of the disclosure as defined by the appended claims. In addition, the disclosure is not limited to the disclosed processes, machines, manufacture, compositions of matter, means, methods, or steps in the particular embodiments set forth in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps. In addition, each claim constitutes an individual embodiment, and the protection scope of the present disclosure also includes each claim scope and a combination of the embodiments.

What is claimed is:

1. A wearable measuring device, comprising:
a wearable element having an opening;
a liquid-transferring element disposed in the wearable element, the liquid-transferring element having a sampling portion exposed to outside through the opening;
a bio-sensing electrode connected to a first position of the liquid-transferring element;
a flow-sensing electrode connected to a second position of the liquid-transferring element; and
a meter disposed on or in the wearable element and connected to the bio-sensing electrode and the flow-sensing electrode.

2. The wearable measuring device as claimed in claim 1, wherein a distance between the second position and the sampling portion is greater than a distance between the first position and the sampling portion.

3. The wearable measuring device as claimed in claim 1, wherein the flow-sensing electrode includes a first conductive component and a second conductive component.

4. The wearable measuring device as claimed in claim 3, wherein a distance between the first conductive component and the sampling portion is greater than a distance between the second conductive component and the sampling portion.

5. The wearable measuring device as claimed in claim 1, wherein the bio-sensing electrode includes a conductive element, and the conductive element includes cobalt, nickel, selenium, cobalt oxide, nickel oxide, selenium oxide, silver carbon, graphene, carbon nanotube, or any combination thereof.

6. The wearable measuring device as claimed in claim 1, wherein the bio-sensing electrode includes a conductive element and a reacting element, the reacting element is between the conductive element and the liquid-transferring element, the reacting element includes a reactant, and the reactant includes immobilized enzymes, enzymes, antibodies, proteins, nucleic acids, chemical reaction reagents or a combination thereof.

7. The wearable measuring device as claimed in claim 6, wherein the bio-sensing electrode further includes a fixing layer for fixing the reactant, and the reactant includes enzymes, antibodies, proteins, nucleic acids, chemical reaction reagents or a combination thereof.

8. A method for measuring biological targets using a wearable measuring device, comprising the following steps:
continuously measuring instantaneous biological target concentrations in a biological liquid using the bio-sensing electrode as claimed in claim 1
continuously measuring instantaneous biological liquid volumes using a flow-sensing electrode; and
calibrating a biological target concentration using the following formula:
$${\sum }_{n = 0}^{t} ( fn ( An ) fn ( Sn ) ) + c ,$$
wherein An represents the instantaneous biological target concentrations, Sn represents the instantaneous biological liquid volumes, t represents a measuring time, and c is a constant.

9. The method as claimed in claim 8, wherein the step of measuring an instantaneous biological target concentration in a biological liquid includes measuring a response current value when the biological liquid is passing through the bio-sensing electrode.

10. The method as claimed in claim 8, wherein the step of measuring instantaneous biological liquid volumes includes measuring current and voltage changes when the biological liquid is passing through the flow-sensing electrode.
